(19) United States
(12) Patent Application Publication
(10) Pub. No.: US 2015/0320812 A1
(43) Pub. Date: Nov. 12, 2015

(54) COMPOSITION AND METHOD TO IMPROVE BLOOD LIPID PROFILES AND REDUCE LOW DENSITY LIPOPROTEIN (LDL) PER-OXIDATION IN HUMANS USING ALGAE BASED OILS AND ASTAXANTHIN

(71) Applicant: U.S. NUTRACEUTICALS, LLC d/b/a Valensa International, Eustis, FL (US)

(72) Inventors: John A. MINATELLI, Mount Dora, FL (US); W. Stephen Hill, Ocala, FL (US); Rudi E. Moerck, Sanford, FL (US)

(21) Appl. No.: 14/806,973

(22) Filed: Jul. 23, 2015

Related U.S. Application Data
(62) Division of application No. 14/219,484, filed on Mar. 19, 2014.
Application No. 14/219,484 is a continuation-in-part of application No. 13/893,572, filed on May 14, 2013, now Pat. No. 8,728,531.
Application No. 13/893,572 is a division of application No. 13/093,201, filed on Apr. 25, 2011, now Pat. No. 8,663,704.
(60) Provisional application No. 61/329,744, filed on Apr. 30, 2010.

(51) Int. Cl. A61K 36/05; A61K 31/122; A23L 1/30; A61K 31/70; A61K 31/66; A61K 36/02; A61K 31/202

(52) U.S. Cl. CPC A61K 36/05; A61K 36/02; A61K 31/122; A61K 31/202; A61K 31/70; A61K 31/66; A23L 1/3008; A23V 2002/00

(57) ABSTRACT

In accordance with a non-limiting example, an algae based oil is used in place of a krill oil to treat low density lipoprotein (LDL) oxidation in humans by administering a therapeutic amount of a dietary supplement composition comprising an algae based oil comprising glycolipids and phospholipids and Eicosapentaenoic (EPA) fatty acids in combination with astaxanthin derived from Haematococcus pluvialis (Hp) in an oral dosage form, wherein the astaxanthin derived from Haematococcus pluvialis (Hp) is 0.1 to 2.7 percent by weight of the algae based oil.

### RELATED APPLICATION(S)

This application is a divisional of application Ser. No. 14/219,484 filed Mar. 19, 2014, which is a continuation-in-part of application Ser. No. 13/893,572, filed May 14, 2013 (U.S. Pat. No. 8,728,531), which is a divisional of application Ser. No. 13/093,201 filed Apr. 25, 2011 (U.S. Pat. No. 8,663,704), which is based on provisional application Ser. No. 61/329,744, filed Apr. 30, 2010, the disclosures which are hereby incorporated by reference in their entirety.

### FIELD OF THE INVENTION

This invention relates to improving blood lipid profiles and reducing low density lipoprotein (LDL) oxidation using therapeutic compositions and methods derived from algae based oil compositions.

### BACKGROUND OF THE INVENTION

The use of krill and/or marine oil are disclosed in U.S. Patent Publication Nos. 2004/0234587; 2004/0241249; and 2007/0098808, the disclosures which are hereby incorporated by reference in their entirety, and discussed in related U.S. patent application Ser. Nos. 12/840,372 and 13/079,238. The beneficial aspects of using krill and/or marine oil are shown also in a research paper published by L. Deutsch as “Evaluation of the Effect of Neptune Krill Oil on Chronic Inflammation and Arthritic Symptoms,” published in the Journal of the American College of Nutrition, Volume 26, No. 1, 39-49 (2007), the disclosure which is hereby incorporated by reference in its entirety.

The published '587, '249 and '808 applications discuss the beneficial aspects of using krill oil in association with pharmaceutically acceptable carriers. As an example, this krill and/or marine oil can be obtained by the combination of detailed steps as taught in the '808 application, by placing krill and/or marine material in a ketone solvent, separating the liquid and solid contents, recovering a first lipid rich fraction from the liquid contents by evaporation, placing the solid contents and organic solvent in an organic solvent of the type as taught in the specification, separating the liquid and solid contents, recovering a second lipid rich fraction by evaporation of the solvent from the liquid contents and recovering the solid contents. The resultant krill oil extract has also been used in an attempt to decrease lipid profiles in patients with hyperlipidemia. The '808 publication gives details regarding this krill oil as derived using those general steps identified above.

The published article gives further details of how the processed krill oil alone, at 3000 mgs/daily dose is a product that aids in treating chronic inflammation and arthritic symptoms. The article describes a study, which had several objectives: a) to evaluate the effect of Neptune Krill Oil on C-reactive protein (C-RP) on patients with chronic inflammation; and b) to evaluate the effectiveness of the Neptune Krill Oil on arthritic symptoms. The method used a randomized, double blind, placebo controlled study protocol. Ninety patients were recruited with either a confirmed diagnosis of cardiovascular disease and/or rheumatoid arthritis and/or osteoarthritis and with increased levels of CRP (>1.0 mg/dl) upon three consecutive weekly blood analysis prior to initiation of oral treatment with krill oil. It is important to note that C-RP is a well known biomarker for risk of cardiovascular disease, therefore in this trial, since patients with known cardiovascular disease states were not excluded from the trial the protocol appears to have evaluated the effects of krill oil on this cardiovascular risk factor while evaluating the effects of krill oil supplementation on the pain and discomfort associated with OA and RH. Group A received the Neptune Krill Oil (300 mg daily) and group B received a placebo. C-RP and Western Ontario and McMaster Universities (WOMAC) osteoarthritis scores were measured at baseline and days 7, 14 and 30. After seven days of treatment, the Neptune Krill Oil reduced CRP by 19.3% compared to an increase by 15.7% observed in the placebo group (p=0.049). After 14 and 30 days of treatment, the Neptune Krill Oil further decreased CRP by 29.7% and 30.9% respectively (p<0.001). The CRP levels of the placebo group increased to 32.1% after 14 days and then decreased to 25.1% at day 30. The between group difference was statistically significant; p=0.004 at day 14 and p=0.008 at day 30. The application of the processed Neptune Krill Oil showed a significant reduction in all three WOMAC scores. After seven days of treatment, the Neptune Krill Oil reduced pain scores by 28.9% (p=0.050), reduced stiffness by 20.3% (p=0.001) and reduced functional impairment by 22.8% (p=0.008). The results of that study indicate that the Neptune Krill Oil at a daily dose of about 300 mg significantly inhibits inflammation, reduces arthritic symptoms within a short treatment period of 7 and 14 days and may be effective in reducing the risk of cardiovascular disease by reduction of C-RP in the patient population employed. It is desirable if further enhanced effects be accomplished using krill oil and similar compositions, especially with improving blood lipid profiles and reducing LDL oxidation.

The commonly assigned and incorporated by reference '572 parent and '201 grandparent applications disclose the beneficial and synergistic effects of improving blood lipid profiles and reducing low density lipoprotein (LDL) oxidation when krill oil and/or marine oil is used in combination with astaxanthin. Although use of krill oil in those applications was one focus, those applications also disclosed that the composition may use fatty acid rich oils derived from algae. A marine based or other algae based oil as an example includes phospholipid and glycolipid bound EPA (Eicosapentaenoic acid) as compared to fish oils that are triacylglycerides. Further development has been accomplished with different algae species that produce EPA alone or EPA and DHA (Docosahexaenoic acid) so that an algae based oil is advantageously used in place of krill oil with the composition and methodology as disclosed in the '572 parent and '201 grandparent applications.

### SUMMARY OF THE INVENTION

In accordance with a non-limiting example, an algae based oil is used in place of a krill oil to treat low density lipoprotein (LDL) oxidation in humans by administering a therapeutic amount of a dietary supplement composition comprising an algae based oil comprising glycolipids and phospholipids and Eicosapentaenoic (EPA) fatty acids in combination with astaxanthin derived from Haematococcus pluvialis (Hp) in an oral dosage form, wherein the astaxanthin derived from Haematococcus pluvialis (Hp) is 0.1 to 2.7 percent by weight of the algae based oil.

The algae based oil is a marine based algae oil in one non-limiting example and includes EPA conjugated with phospholipid and glycerolipid as glycolipid bound polar lipids. It may include DHA also conjugated with the phospholipid and glycolipid bound polar lipids. In one example, the algae based oil has an EPA titre higher than DHA and contains phospholipid and glycolipid bound EPA as compared to fish oils that are triacylglycerides. Different algae species may be used that produce EPA alone or EPA and DHA, including those algae species such as nannochloropsis oculata for EPA production and various diatom species of microalgae.

In one example the astaxanthin derived from Haematococcus pluvialis (Hp) is 0.4 to 0.67 percent by weight of the algae based oil. In yet another example, the algae based oil includes Eicosapentaenoic (EPA) and Docosahexaenoic (DHA) fatty acids in the form of glycolipids and phospholipids. In one non-limiting example, the algae based oil may be formed from 5-10 percent phospholipids and 35-40 percent glycolipids. In yet another example, the algae based oil includes at least 15 percent EPA. The EPA fatty acids are conjugated with phospholipid and glycolipid polar lipids in an example.

In an example, the algae based oil may be derived from the microalgae Nannochloropsis oculata comprising Eicosapentaenoic (EPA) fatty acids in the form of glycolipids and phospholipids. The algae based oil may also be derived from the microalgae selected from the group consisting of thalassiosira sp., tetraselmis sp., chaetoceros sp., and isochrysis sp., and comprising Eicosapentaenoic (EPA) and Docosahexaenoic (DHA) fatty acids in the form of glycolipids and phospholipids.

The algae based oil may be derived from the microalgae selected from the group consisting of grateloupia turuturu; porphyridium cruentum; monodus subterraneus; phaeodactylum tricornutum; isochrysis galbana; navicula sp.; pythium irregulare; nannochloropsis sp.; and nitzschia sp. and comprising Eicosapentaenoic (EPA) and Docosahexaenioic (DHA) fatty acids in the form of glycolipids and phospholipids.

The algae based oil may be derived from the microalgae selected from the group consisting of Asterionella japonica, Bidulphia sinensis, Chaetoceros septentrionale, Lauderia borealis, Navicula biskanteri, Navicula laevis (heterotrof.), Navicula laevis, Navicula incerta, Stauroneis amphioxys, Navicula pellicuolsa, Bidulphia aortia, Nitzschia alba, Nitzschia chosterium, Phaeodactylum tricornutum, Phaeodactylum tricornutum, Skeletonema costatum, Pseudopedinella sp., Cricosphaera elongate, Monodus subterraneus, Nannochloropsis, Rodela violacea 115.79, Porphyry. Cruentum 1380.Id, Pavlova salina, Cochlodinium heteroloblatum, Cryptecodinium cohnii, Gonyaulax catenella, Gyrodinium cohnii, Prorocentrum minimum, Chlorella minutissima, Isochrysis galbana ALII4, Phaeodactylum tricornutum WT, Porphyridium cruentum, and Monodus subterraneus and comprising Eicosapentaenoic (EPA) and Docosahexaenoic (DHA) fatty acids in the form of glycolipids and phospholipids.

The algae based oil may be derived from a fungi selected from the group consisting of Mortierella Mortierella alpine IS-4, and Pythium irregulare, or a bacteria as SCRC-2738 and comprising Eicosapentaenoic (EPA) and Docosahexaenoic (DHA) fatty acids in the form of glycolipids and phospholipids. In an example, 1-4000 mg of algae based oil per daily dose may be delivered and in yet another example 0.1-12 mg astaxanthin supplemented to the algae based oil per daily dose may be delivered. The astaxanthin in an example is derived from Haematococcus pluvialis algae oleoresin or beadlet.

### DETAILED DESCRIPTION OF THE PREFERRED EMBODIMENTS

The present invention will now be described more fully hereinafter in which preferred embodiments of the invention are described. This invention may, however, be embodied in many different forms and should not be construed as limited to the embodiments set forth herein. Rather, these embodiments are provided so that this disclosure will be thorough and complete, and will fully convey the scope of the invention to those skilled in the art.

It is known that algae can be an important source for omega-3 fatty acids such as EPA and DHA. It is known that fish and krill do not produce omega-3 fatty acids but accumulate those fatty acids from the algae they consume. Omega-3 bioavailability varies and is made available at the site of physiological activity depending on what form it is contained. For example, fish oil contains omega-3 fatty acids in a triglyceride form that are insoluble in water and require emulsification by bile salts via the formation of micelles and subsequent digestion by enzymes and subsequent absorption. Those omega-3 fatty acids that are bound to polar lipids, such as phospholipids and glycolipids, however, are not dependent on bile for digestion and go through a simpler digestion process before absorption. Thus, these omega-3 fatty acids, such as from an algae based oil, have greater bioavailability for cell growth and functioning as compared to the omega-3 triglycerides of fish oil. There are many varieties of algae that contain EPA conjugated with phospholipid and glycolipid polar lipids or contain EPA and DHA conjugated with phospholipids and glycolipids.

Throughout this description, the term “algae” or “microalgae” may be used interchangeably to each other with microalgae referring to photosynthetic organisms that are native to aquatic or marine habitats and are too small to be seen easily as individual organisms with the naked eye. When the term “photoautotropic” is used, it refers to growth with light as the primary source of energy and carbon dioxide as the primary source of carbon. Other forms of biomass that may encompass algae or microalgae may be used and the term “biomass” may refer to a living or recently dead biological cellular material derived from plants or animals. The term “polar” may refer to the compound that has portions of negative and/or positive charges forming negative and/or positive poles. The term “oil” may refer to a combination of fractionable lipid fractions of a biomass. As known to those skilled in the art, this may include the entire range of various hydrocarbon soluble in non-polar solvents and insoluble, or relatively insoluble in water as known to those skilled in the art. The microalgae may also include any naturally occurring species or any genetically engineered microalgae to have improved lipid production.

There now follows a description of the dietary supplement composition and associated method used to improve blood lipid profiles and reduce low density lipoprotein (LDL) per-oxidation in humans as based on the original disclosure in the '572 and '201 applications and related to the krill oil and marine based algae oil disclosed in these applications, followed by further details of the algae based oil and its composition and use relative to an algae based oil having phospholipid and glycolipid bound EPA or EPA and DHA. The algae based oil is substituted for the previous krill oil in those applications. The description will begin, however, with details of the previous disclosure in the parent and grandparent applications regarding the use of krill oil included in the composition, and then proceed with the description and details of the algae based oil. It should be understood that when krill oil is specifically described and its composition as related to the disclosure in the '572 and '201 parent and grandparent applications described, the algae based oil may be substituted therefor. Some of the composition components will change such as the levels of EPA and/or EPA and DHA and other components when the algae based oil is used as later shown in various tables within the following description.

The composition as related to the krill oil disclosure in the parent and grandparent applications includes EPA and DHA functionalized as marine phospholipids and acyltriglycerides derived from krill. Both the krill and algae based oil, in accordance with a non-limiting example, however, may include esterified astaxanthin. It has been found that a new and potentially quite important biomarker for cardiovascular risk is related to the amount of EPA and DHA found in red blood cells divided by the total fatty acid content in red blood cells or the so called “omega-3 index.” The compositions, in accordance with a non-limiting example, improve the omega-3 index in man on prolonged administration and therefore are presumed to lower cardiovascular event risks. Some of these components as related to the krill oil are explained in the following chart:

| Components | Percentage (%) |
|---|---|
| PHOSPHOLIPIDS | |
| PC, PE, PI, PS, SM, CL | >40 |
| OMEGA-3 (functionalized on PL) | >30 |
| Eicosapentaenoid Acid (EPA)* | >17 (15% in one example and |
| | 10% in another) |
| Docosahexaenoid Acid (DHA)+ | >11 (9% in one example and |
| | 5% in another) |
| ANTIOXIDANTS | (mg/100 g) |

Astaxanthin, Vitamin A, Vitamin E >1.25
*>55% of PL-EPA/Total EPA
+>55% of PL-DHA/Total DHA

These amounts can vary depending on application and persons.

The krill oil or algae based oil is supplemented with astaxanthin to improve formulated product utility. In one study, 4 mg of astaxanthin per day for two weeks resulted in a 26% reduction of LDL cholesterol oxidation. 4 mg of astaxanthin for eight weeks resulted in a 21% decrease in C-reactive protein scores. 3.6 mg of astaxanthin per day for two weeks demonstrated that astaxanthin protects LDL cholesterol against induced in vitro oxidation.

Astaxanthin is also known to reduce C-Reactive Protein (C-RP) blood levels in vivo. For example, in human subjects with high risk levels of C-RP three months of astaxanthin treatment resulted in 43% drop in the patient population's serum C-RP levels a drop which is below the unacceptable cardiovascular event risk level. Astaxanthin is so powerful that it has been shown to negate the pro-oxidant activity of Vioxx in vitro, a COX-2 inhibitor belonging to the NSAIDS drug class which is known to cause cellular membrane lipid per-oxidation leading to heart attacks and strokes. For this reason Vioxx was subsequently removed from the US market by the FDA. Astaxanthin is also absorbed in vitro by lens epithelial cells where it suppresses UVB induced lipid per-oxidative mediated cell damage at umol/L concentrations. Reduction of C-Reactive protein (CRP), reduction of LDL oxidation and an increase in the omega-3 index in vivo would presumably all be important positive contributors to cardiovascular health since each are well know biomarkers for cardiovascular health risk. These results have been shown in:

1) Lee et al., Molecules and Cells, 16(1):97-105; 2003;

2) Ohgami et al., Investigative Ophthalmology and Visual Science 44(6):2694-2701, 2003;

3) Spiller et al., J. of the Amer, College of Nutrition, 21(5): October 2002; and

4) Harris, Pharmacol. Res. 2007 March; 55(3) 217-223.

A preferred composition in one embodiment includes 300-500 mg of krill oil or an algae based oil and 2 mg astaxanthin. Up to 8 mg and possibly 12 mg may be used in some examples.

As noted before, krill oil is typically produced from Antarctic krill (euphausia superba), which is a zooplankton (base of food chain). It is one of the most abundant marine biomass of about 500 million tons according to some estimates. Antarctic krill breeds in the pure uncontaminated deep sea waters. It is a non-exploited marine biomass and the catch per year is less than or equal to about 0.02% according to some estimates.

It is believed that Krill oil based phospholipid bound EPA and DHA uptake into cellular membranes is far more efficient than triacylglyercide bound EPA and DHA since liver conversion of triacylglycerides is itself inefficient and because phospholipid bound EPA and DHA can be transported into the blood stream via the lympathic system, thus, avoiding liver breakdown. In addition, krill oil consumption does not produce the burp-back observed with fish oil based products. Because of this burp-back feature of fish oils, it has been found that approximately 50% of all consumers who try fish oil never buy it again.

Astaxanthin has an excellent safety record. A conducted study obtained the results as follows:

Oral LD 50: 600 mg/kg (rats);

NOAEL: 465 mg/kg (rats); or

Serum Pharmacokinetics: Stewart et al. 2008

1) T_{1/2}: 16 hours;

2) Tₘₐₓ: 8 hours;

3) Cₘₐₓ: 65 μg/L.

At eight weeks of supplementation at 6 mg per day, there was no negative effect in healthy adults. Spiller et al. 2003.

In accordance with one non-limiting example, astaxanthin has three prime sources. 3 mg astaxanthin per 240 g serving of non-farmed raised salmon or a 1% to 12% astaxanthin oleoresin or 1.5-2.5% beadlet derived from microalgae. Literature references pertinent to the above discussion can be found in Lee et al., Molecules and Cells 16(1): 97-105, 2003; Ohgami et al., Investigative Ophthalmology and Visual. Science 44(6): 2694-2701, 2003; Spiller et al., J. of the American College of Nutrition 21(5): October 2002; and Fry et al., University of Memphis, Human Performance Laboratories, 2001 and 2004, Reports 1 and 2.

Many beneficial and synergistic effects are now being reported herein have been observed when krill oil is used in combination with other active ingredients, and more specifically in one example, krill oil in combination with astaxanthin. It should be understood that different proportions of ingredients and percentages in compositions can be used depending on end use applications and other environmental and physiological factors when treating a patient condition.

The krill oil in one example is derived from Euphasia spp., comprising Eicosapentaenoic (EPA) and Docosahexaenoic (DHA) fatty acids in the form of triacylglycerides and phospholipids, although not less than 1% EPA and 5% DHA has been found advantageous. In another example, the krill oil includes at least 15% EPA and 9% DHA, of which not less than 45% are in the form of phospholipids, and in one example, greater than 50%. The composition can be delivered advantageously for therapeutic results with 1-4000 mg of krill oil delivered per daily dose. In another example, 0.1-50 mg astaxanthin are supplemented to the krill oil per daily dose, and in one example, 0.1-12 mg of astaxanthin.

The astaxanthin is preferably derived from Haematococcus pluvialis algae, Pfaffia, krill, or by synthetic routes, in the known free diol, monoester or diester form, and in one example, at a daily dose of 0.5-8 mg. When this amount of astaxanthin, especially as derived from Haematococcus pluvialis, is applied to the range of 300-500 mg of krill oil or algae based oil, the numerical range of about 0.1 to 2.7 percent by weight of the krill oil or algae based oil is obtained.

The composition may also include an n-3 (omega-3) fatty acid rich oil derived from fish oil, algae oil, flax seed oil, or chia seed oil when the n-3 fatty acid comprises alpha-linolenic, stearidonic, eicosapentaenoic or docosapentaenoic acid. The composition may include naturally-derived and synthetic antioxidants that are added to retard degradation of fatty acids and astaxanthin.

Details of a type of CO2 extraction and processing technology (as supercritical CO2 extraction) and peroxidation blacker technology that can be used are disclosed in commonly assigned U.S. Patent Publication Nos. 2009/0181127; 2009/0181114; and 2009/0258081, the disclosures which are hereby incorporated by reference in their entirety.

As noted before, there are beneficial aspects of using krill oil or algae based oil in synergistic combination with other ingredients. It has been determined that a fish oil derived, choline based, phospholipid bound omega-3 fatty acid mixture including phospholipid bound polyunsaturated EPA and DHA is also advantageous for improving blood lipid profiles and reducing LDL either alone or admixed with other ingredients, for example, an LDL per-oxidation blocker. One commercially available example of a mixture of fish oil derived, choline based, phospholipid bound fatty acid mixture including polyunsaturated EPA and DHA is Omega Choline 1520F as a phospholipid, omega-3 preparation, which is derived from natural fish oil and sold by Enzymotec Ltd. One example of such composition is described below:

Ingredients (g/100 g):

| Pure Marine Phospholipids | n.l.t. 15 |
|---|---|
| DHA* | n.l.t. 12 |
| EPA** | n.l.t. 7 |
| Omega-3 | n.l.t. 22 |
| Omega-6 | <3 |

*Docosahexaenoic acid
**Eicosapenteanoic acid

### Analytical Data:

| | |
|---|---|
| Peroxide value (meq/Kg) | n.m.t. 5 |
| Loss on Drying (g/100 g) | n.m.t. 2 |

### Physical Properties:

| | |
|---|---|
| Consistency | Viscous Liquid |

In accordance with a non-limiting example, the method improves blood lipid profiles and either alone or in combination with added astaxanthin, such as a per-oxidation blocker, and reduces LDL oxidation in a patient by administering a therapeutic amount of a composition including a mixture of fish oil derived, choline based, phospholipid bound omega-3 fatty acid mixture including phospholipid bound polyunsaturated EPA and DHA either alone or admixed with an LDL per-oxidation blocker such as astaxanthin. In one example, the composition is supplemented in combination with astaxanthin in an oral dosage form. The mixture of fish oil derived, choline based, phospholipid bound fatty acid mixture including polyunsaturated EPA and DHA in one example comprises Eicosapentaenoic (EPA) and Docosahexaenoic (DHA) fatty acids in the form of triacylglycerides and phospholipids. In another example, the omega choline includes at least 7% EPA and 12% DHA, of which not less than 15% are in the form of phospholipids. The composition can be delivered advantageously for therapeutic results with 1-4000 mg of a mixture of fish oil and fish oil derived, choline based, phospholipid bound fatty acid mixture including polyunsaturated EPA and DHA delivered per daily dose. In another example, 0.1-20 mg astaxanthin are supplemented to the Omega Choline per daily dose.

It should be understood that an instant formulation can be used for LDL reduction using only a mixture of fish oil derived, choline based, phospholipid bound fatty acid mixture including polyunsaturated EPA and DHA. It is also possible to use a mixture of fish oil derived, choline based, phospholipid bound omega-3 fatty acid mixture (including polyunsaturated EPA and DHA) mixed with astaxanthin. It should also be understood that an enriched version of a mixture of fish oil derived, choline based, phospholipid bound fatty acid mixture including polyunsaturated EPA and DHA can be used wherein the fraction of added fish oil diluents has been decreased and the proportion of fish oil derived phospholipids has been increased. This can be accomplished by using supercritical CO2 and/or solvent extractions for selective removal of triacylglycerides from phospholipids. The composition may also include a natural or synthetic cyclooxygenase-1 or -2 inhibitor comprising for example aspirin, acetaminophen, steroids, prednisone, or NSAIDs. The composition may also include a gamma-linoleic acid rich oil comprising Borage (Borago officinalis L.) or Safflower (Carthamus tinctorius L.), which delivers a metabolic precursor to PGE₁ synthesis.

The composition may also include an n-3 (omega-3) fatty acid rich oil derived from fish oil, algae oil, flax seed oil, chia seed oil or perilla seed oil wherein the n-3 fatty acid source comprises alpha-linolenic, stearidonic, eicosapentaenoic or docosapentaenoic acid. The composition may include naturally-derived and synthetic antioxidants that are added to retard degradation of fatty acids such as tocopherols, tocotrienols, carnosic acid or Carnosol and/or astaxanthin.

As noted above, algae based oil is used and substituted for the krill oil in an example. This algae based oil may be substituted for krill oil and provide an algae sourced EPA or an EPA/DHA based oil in which oils are present in phospholipid and glycerolipid forms, as glycolipids. Different algae based oils derived from different microalgae may be used. One preferred example algae based oil has the EPA titre higher than the DHA as compared to a class of omega-3's from fish oils that are triacylglycerides. These algae based oils are rich in EPA and in the phospholipid and glycolipid forms. An example marine based algae oil is produced by Parry Nutraceuticals as a division of EID Parry (India) Ltd. as an omega-3 (EPA) oil.

The following first table shows the specification of an algae based oil as manufactured by Parry Nutraceuticals identified above, followed by a second table for a fatty acid profile chart of that algae based oil. A third table is a comparative chart of the fatty acid profiles for non-algae based oils. These charts show that the algae based oil has a high EPA content of phospholipids and glycolipids. The algae based oils may be processed to enrich selected constituents using supercritical CO2 and/or solvent extractions as noted above and other techniques.

SPECIFICATION: ALGAE BASED OIL

| | | | TEST |
|---|---|---|---|
| | SPECIFICA- | | METHOD/ |
| PARAMETERS | TION | SOP. NO | REFERENCE |
| Physical Properties | | | |
| Appearance | Viscous oil | QA - 88 | In house |
| Color | Brownish black | QA - 88 | In house |
| Odor | Characteristic | QA - 88 | In house |
| Taste | Characteristic | QA - 88 | In house |
| General Composition | | | |
| Loss on drying (%) | 2.0-3.0 | QA - 038 | USP <731> |
| | | | Loss on |
| | | | drying |
| Ash (%) | 0.5-1.0 | QA - 080 | AOAC |
| | | | Official |
| | | | Method |
| | | | 942.05, 16th |
| | | | Edition |
| Protein (%) | 1.0-2.0 | QA - 021 | AOAC |
| | | | Official |
| | | | method |
| | | | 978.04, 16th |
| | | | Edn. |
| Carbohydrate (%) | 1.0-2.0 | | AOAC |
| | | | 18th Edn |
| | | | 2006/By |
| | | | Difference |
| Residual Solvent | NMT 100 | QA - 074 | GC - Head |
| (ppm) (as Ethyl | | | Space, |
| Acetate) | | | |
| (as Acetone) | NMT 30 | | USP <467) |
| Lipid Composition | | | |
| Total Lipid (%) | 92.0-95.0 | QA - 86 | AOAC |
| | | | official |
| | | | method |
| | | | 933.08 |
| Chlorophyll (%) | NMT 1.50 | QA - 078 | Jeffrey & |
| | | | Humphrey |
| | | | (1975) - |
| | | | Photo- |
| | | | synthetic |
| | | | pigments |
| | | | of Algae |
| | | | (1989) |
| Total carotenoids (%) | NMT 1.50 | QA - 85 | By JHFA |
| | | | method- 1986 |
| Total | NMT 12.0 | QA - 086 | AOAC |
| Unsaponifiables (%) | | | official |
| | | | method |
| | | | 933.08 |
| Omega 3 [EPA + | NLT 15.00 | QA - 087 | In House |
| DHA] - % w/w | | | method |
| Total Omega 3 (% w/w) | NLT 17.00 | | |
| Total Omega 6 (% w/w) | NMT 5.00 | | |
| Total EFA (% w/w) | NLT 20. | | |
| Lipid percentage | | | |
| Triglycerides | 15-20% | | |
| Phospholipids | 5-10% | | |
| Glycolipids | 35-40% | | |
| Free fatty acids | 15-20% | | |
| Microbial parameters | | QA - 039 | AOAC, 1995, |
| | | | Chapter 17 |
| Standard Plate Count | NMT 1,000 | | |
| (cfu/1 g) | | | |
| Yeast & Mold (cfu/1 g) | NMT 100 | | |
| Coli forms (/10 g) | Negative | | |
| E. coli (/10 g) | Negative | | |
| Staphylococcus (/10 g) | Negative | | |
| Salmonella (/10 g) | Negative | | |
| Fatty acid profile | | | |
| (Area %) | | | |
| Myristic acid [14.0] | NLT 4.0 | QA - 086 & | In House GC |
| | | 087 | method |
| Palmiltic acid [16:0] | NLT 16.0 | | |
| Palmito oleic acid | NLT 12.0 | | |
| [16:1, n-9] | | | |
| Hexadecadienoic acid | NLT 4.0 | | |
| [16:2, n-4] | | | |
| Hexadecatrienoic acid | NLT 12.0 | | |
| [16:3, n-4] | | | |
| Stearic acid [18:0] | NLT 0.10 | | |
| Oleic acid [18:1] | NLT 1.0 | | |
| Linoleic acid | NLT 1.0 | | |
| [18:2, n-6] - LA | | | |
| Alpha Linolenic acid | NLT 0.50 | | |
| [18:3, n-3] - ALA | | | |
| Stearidonic acid | NLT 0.10 | | |
| [18:4, n-3] - SA | | | |
| Arachidonic Acid | NLT 0.25 | | |
| [20:4, n-6] - AA | | | |
| Eicosapentaenoic acid | NLT 15.0 | | |
| [20:5, n-3] | | | |
| Decosahexaenoic acid | NLT 1.5 | | |
| [20:6, n-3] | | | |
| Heavy Metals | | | |
| Lead (ppm) | NMT 1.0 | External | AOAC 18th |
| Arsenic (ppm) | NMT 0.5 | lab | Edn: 2006 |
| Cadmium (ppm) | NMT 0.05 | reports | By ICPMS |
| Mercury (ppm) | NMT0.05 | | |

Safety: Safe for the intended use
Shelf life: 24 months from the date of manufacture
Stability: Stable in unopen conditions
Storage: Store in a cool, dry place away from sunlight, flush container with Nitrogen after use
Documentation: Every Batch of shipment carries COA
Packing: 1 kg, 5 kg, and 20 kg food grade containers

FATTY ACID PROFILE CHART
ALGAE BASED OIL

| | ALGAE BASED |
|---|---|
| | OMEGA-3 |
| FATTY ACID | (EPA) OIL |
| Total fatty acid, gm/100 gm of oil | 75 gm |
| Fatty acid [% of total fatty acid] | |
| Myristic acid [14:0] | 6.87 |
| Pentadecanoic acid [15:0] | NA |
| Palmitic acid [16:0] | 20.12 |
| Palmito oleic acid [16:1, ω-9] | 18.75 |
| Hexadeca dienoic acid [16:2, ω-4] | 6.84 |
| Hexadeca trienoic acid [16:4, ω-4] | 12.54 |
| Heptadecanoic acid [17:0] | NA |
| Stearic acid [18:0] | 0.68 |
| Oleic acid [18:1, ω-9] | 3.56 |
| Linoleic acid [18:2, ω-6] | 2.68 |
| Alpha linolenic acid [18:3, ω-3] | 3.73 |
| Gamma linolenic acid [18:3, ω-6] | NA |
| Stearidonic acid [18:4, ω-3] | 0.33 |
| Arachidonic acid [20:4, ω-6] | 0.97 |
| Eicosapentaenoic acid [20:5, ω-3] EPA | 23.00 |
| Docosapentaenoic acid [22:5, ω-3] DHA | NA |
| Docosahexaenoic acid [22:6, ω-3] DHA | 3.26 |
| others | 3.54 |
| EPA/DHA [gm/100 gm oil] | 15.75 |
| Total ω-3 fatty acids [gm/100 gm oil] | 18.20 |
| LIPD CLASS DETAILS [gm/100 gm oil] | |
| Unsaponifiables [carotenoids, chlorophyll, | 12 |
| sterol, fatty alcohol etc.,] | |
| Free fatty acids | 20 |
| Triglydcerides | 20 |
| Phospholipids | 10 |
| Glycolipids | 38 |
| Total | 100 |
| STABILITY [months] | 24 |

FATTY ACID PROFILE - COMPARATIVE CHART
NON-ALGAE BASED OILS

| | FISH OIL | KRILL | MARTEK |
|---|---|---|---|
| FATTY ACID | MAXEPA | OIL | OIL |
| Total fatty acid, gm/100 gm of oil | 95 gm | 70-80 gm | 95 gm |
| Fatty acid [% of total fatty acid] | | | |
| Myristic acid [14:0] | 8.68 | 11.09 | 11.47 |
| Pentadecanoic acid [15:0] | NA | NA | NA |
| Palmitic acid [16:0] | 20.35 | 22.95 | 26.36 |
| Palmito oleic acid | 11.25 | 6.63 | NA |
| [16:1, ω-9] | | | |
| Hexadecadienoic acid | NA | NA | NA |
| [16:2, ω-4] | | | |
| Hexadecatrienoic acid | NA | NA | NA |
| [16:4, ω-4] | | | |
| Heptadecanoic acid [17:0] | NA | NA | NA |
| Stearic acid [18:0] | 4.67 | 1.02 | 0.50 |
| Oleic acid [18:1, ω-9] | 13.07 | 17.93 | 1.50 |
| Linoleic acid [18:2, ω-6] | 1.28 | 0.14 | 0.61 |
| Alpha linolenic acid | 0.33 | 2.11 | 0.40 |
| [18:3, ω-3] | | | |
| Gamma linolenic acid | NA | NA | NA |
| [18:3, ω-6] | | | |
| Stearidonic acid | 1.69 | 7.01 | 0.33 |
| [18:4, ω-3] | | | |
| Arachidonic acid | 0.50 | NA | NA |
| [20:4, ω-6] | | | |
| Eicosapentaenoic acid | 20.31 | 19.04 | 1.0 |
| [20:5, ω-3] EPA | | | |
| Docosapentaenoic acid | NA | NA | 15.21 |
| [22:5, ω-3] DHA | | | |
| Docosahexaenoic acid | 13.34 | 11.94 | 42.65 |
| [22:6, ω-3] DHA | | | |
| others | 4.53 | 0.14 | NA |
| EPA/DHA [gm/100 gm oil] | 31.96 | 21.68 | 41.46 |
| Total ω-3 fatty acids | 33.85 | 28.00 | 41.60 |
| [gm/100 gm oil] | | | |
| LIPD CLASS DETAILS | | | |
| [gm/100 gm oil] | | | |
| Unsaponifiables | 5 | 5 | 5 |
| [carotenoids, chlorophyll, | | | |
| sterol, fatty alcohol etc.,] | | | |
| Free fatty acids | 0.5 | 30 | 0.5 |
| Triglydcerides | 94.5 | 25 | 94.5 |
| Phospholipids | Nil | 40 | Nil |
| Glycolipids | Nil | Nil | Nil |
| Total | 100 | 100 | 100 |
| STABILITY [months] | 12 | 24 | 6 |

Different types of marine based algae oils may be used, including nannochloropsis oculata as a source of EPA. Another algae that may be used is thalassiosira weissflogii such as described in U.S. Pat. No. 8,030,037 assigned to the above-mentioned Parry Nutraceuticals, a Division of EID Parry (India) Ltd., the disclosure which is hereby incorporated by reference in its entirety. Other types of algae as disclosed include chaetoceros sp. or prymnesiophyta or green algae such as chlorophyta and other microalgae that are diamons tiatoms. The chlorophyta could be tetraselmis sp. and include prymnesiophyta such as the class prymnesiophyceae and such as the order isochrysales and more specifically, isochrysis sp. or pavlova sp.

There are many other algae species that can be used to produce EPA and DHA as an algae based oil whether marine based or not to be used in accordance with a non-limiting example. In some cases, the isolation of the phospholipid and glycolipid bound EPA and DHA based oils may require manipulation of the algae species growth cycle.

Other algae/fungi phospholipid/glycolipid sources include: grateloupia turuturu; porphyridium cruentum; monodus subterraneus; phaeodactylum tricornutum; isochrysis galbana; navicula sp.; pythium irregule; nannochloropsis sp.; and nitzschia sp.

Details regarding grateloupia turuturu are disclosed in the article entitled, “Grateloupia Turuturu (Halymeniaceae, Rhodophyta) is the Correct Name of the Non-Native Species in the Atlantic Known as Grateloupia Doryphora,” Eur. J. Phycol. (2002), 37: 349-359, as authored by Brigitte Gavio and Suzanne Fredericq, the disclosure which is incorporated by reference in its entirety.

Porphyridium cruentum is a red algae in the family porphyridiophyceae and also termed rhodophyta and is used as a source for fatty acids, lipids, cell-wall polysaccharides and pigments. The polysaccharides of this species are sulphated. Some porphyridium cruentum biomass contains carbohydrates of up to 57%.

Monodus subterraneus is described in an article entitled, “Biosynthesis of Eicosapentaenoic Acid (EPA) in the Fresh Water Eustigmatophyte Monodus Subterraneus (Eustigmatophyceae),” J. Phycol, 38, 745-756 (2002), authored by Goldberg, Shayakhmetova, and Cohen, the disclosure which is incorporated by reference in its entirety. The biosynthesis of PUFAs from algae is complicated and the biosynthesis from this algae is described in that article.

Phaeodactylum tricornutum is a diatom and unlike most diatoms, it can grow in the absence of silicon and the biogenesis of silicified frustules is facultative.

Isochrysis galbana is a microalgae and used in the bivalve aquaculture industry.

Navicula sp. is a boat-shaped algae and is a diatom. Pythium irregule is a soilborne pathogen found on plant hosts.

Nannochloropsis sp. occurs in a marine environment, but also occurs in fresh and brackish water. The species are small, nonmotile spheres that do not express any distinct morphological feature. These algae have chlorophyll A and lack chlorophyll B and C. They can build high concentrations of pigment such as astaxanthin, zeaxanthin and canthaxinthin. They are about 2-3 micrometers in diameter. They may accumulate high levels of polyunsaturated fatty acids.

Nitzschia sp. is a pinnate marine diatom and usually found in colder waters and associated with both Arctic and Antarctic polar sea ice where it is a dominant diatom. It produces a neurotoxin known as domoic acid which is responsible for amnesic shell fish poisoning. It may grow exponentially at temperatures between −4 and −6 degrees C. It may be processed to form and extrapolate the fatty acids.

As a source of polyunsaturated fatty acids, microalgae competes with other micro-organisms such as fungi and bacteria. There may be some bacterial strains that could be an EPA source, but microalgae has been found to be a more adequate and readily available source. Microalgae is a good source of oil and EPA when derived from phaeodactylum, isochrysis and monodus. The microalgae phaeodactylum tricornutum produces a high proportion of EPA. Other different strains and species of microalgae, fungi and possibly bacteria that can be used to source EPA include the following:

### I. Diatoms

Asterionella japonica

Bidulphia sinensis

Chaetoceros septentrionale

Lauderia borealis

Navicula biskanteri

Navicula laevis (heterotrof.)

Navicula laevis

Navicula incerta

Stauroneis amphioxys

Navicula pellicuolsa

Bidulphia aurtia

Nitzschia alba

Nitzschia chosterium

Phaeodactylum tricornutum

Phaeodactylum tricornutum

Skeletonema costatum

### II. Chrysophyceae

Pseudopedinella sp.

Cricosphaera elongate

### III. Eustigmatophyceae

Monodus subterraneus

Nannochloropsis

### IV. Prymnesiophyceae

Rodela violacea 115.79

Porphyry. Cruentum 1380.Id

### V. Prasinophyceae

Pavlova salina

### VI. Dinophyceae

Cochlodinium heteroloblatum

Cryptecodinium cohnii

Gonyaulax catenella

Gyrodinium cohnii

Prorocentrum minimum

### VII. Other Microalgae

Chlorella minutissima

Isochrysis galbana ALII4

Phaeodactylum tricornutum WT

Porphyridium cruentum

Monodus subterraneus

### VIII. Fungi

Mortierella alpine

Mortierella alpine IS-4

Pythium irregulare

### IX. Bacteria

SCRC-2738

Different microalgae may be used to form the algae based oil comprising glycolipids and phospholipids and at least EPA and/or EPA/DHA. Examples include: Chlorophyta, Cyanophyta (Cyanobacteria), and Heterokontophyta. The microalgae may be from one of the following classes: Bacillariophyceae, Eustigmatophyceae, and Chrysophyceae. The microalgae may be from one of the following genera: Nannochloropsis, Chlorella, Dunaliella, Scenedesmus, Selenastrum, Oscillatoria, Phormidium, Spirulina, Amphora, and Ochromonas.

Other non-limiting examples of microalgae species that may be used include: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella saliva, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chiorococcum infusionum, Chiorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Effipsoidon sp., Euglena spp., Franceia sp., Fragilaria crotonensis, Fragilaria sp., Gleocapsa sp., Gloeothamnion sp., Haematococcus pluvialis, Hymenomonas sp., Isochrysis aff. galbana, Isochrysis galbana, Lepocinclis, Micractinium, Micractinium, Monoraphidium minutum, Monoraphidium sp., Nannochioris sp., Nannochioropsis salina, Nannochioropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana. Preferably, the microalgae are autotrophic.

It is also possible to form the oil comprising glycolipids and phospholipids and at least EPA from genetically modified yeast. Non-limiting examples of yeast that can be used include: Cryptococcus curvatus, Cryptococcus terricolus, Lipomyces starkeyi, Lipomyces lipofer, Endomycopsis vernalis, Rhodotorula glutinis, Rhodotorula gracilis, Candida 107, Saccharomyces paradoxus, Saccharomyces mikatae, Saccharomyces bayanus, Saccharomyces cerevisiae, any Cryptococcus, C. neoformans, C. bogoriensis, Yarrowia lipolytica, Apiotrichum curvatura, T. bombicola, T. apicola, T. petrophilum, C. tropicalis, C. lipolytica, and Candida albicans. It is even possible to use a biomass as a wild type or genetically modified fungus. Non-limiting examples of fungi that may be used include Mortierella, Mortierrla vinacea, Mortierella alpine, Pythium debaryanum, Mucor circinelloides, Aspergillus ochraceus, Aspergillus terreus, Pennicillium iilacinum, Hensenulo, Chaetomium, Cladosporium, Maibranchea, Rhizopus, and Pythium.

It is also possible that bacteria may be used that includes lipids, proteins, and carbohydrates, whether naturally occurring or by genetic engineering. Non-limiting examples of bacteria include: Escherichia coli, Acinetobacter sp. any actinomycete, Mycobacterium tuberculosis, any streptomycete, Acinetobacter calcoaceticus, P. aeruginosa, Pseudomonas sp., R. erythropolis, N. erthopolis, Mycobacterium sp., B., U. zeae, U. maydis, B. lichenformis, S. marcescens, P. fluorescens, B. subtilis, B. brevis, B. polmyma, C. lepus, N. erthropolis, T. thiooxidans, D. polymorphis, P. aeruginosa and Rhodococcus opacus.

Possible algae sourced, EPA/DHA based oils that are derived from an algae and contain glycol and phospholipid bound EPA and/or EPA/DHA and may include a significant amount of free fatty acids, triglycerides and phospholipids and glycolipids in the range of 35-40% or more of total lipids are disclosed in the treatise “Chemicals from Microalgae” as edited by Zvi Cohen, CRC Press, 1999. Reference is also made to a study in men that have been given a single dose of oil from a polar-lipid rich oil from the algae nannochloropis oculata as a source of EPA and described in the article entitled, “Acute Appearance of Fatty Acids in Human Plasma—A Comparative Study Between Polar-Lipid Rich Oil from the Microalgae Nannochloropis Oculata in Krill Oil in Healthy Young Males,” as published in Lipids in Health and Disease, 2013, 12:102 by Kagan et al. The EPA in that algae oil was higher than that of krill oil by about 25.06 to 13.63 for fatty acid composition as the percent of oil. The algae oil was provided at 1.5 grams of EPA and no DHA as compared to krill oil that was provided at 1.02 grams EPA and 0.54 grams DHA. The participants consumed both oils in random order and separated by seven days and the blood samples were collected before breakfast and at several time points up to 10 hours after taking the oils.

The researchers determined that the algae based oil had a greater concentration of EPA and plasma than krill oil with the EPA concentration higher with the algae based oil at 5, 6, 8 and 10 hours (P<0.05) intended to be higher at 4 hours (P=0.094). The maximum concentration (CMAX) of EPA was higher with algae oil than with krill oil (P=0.010). The maximum change in concentration of EPA from its fasting concentration was higher than with krill oil (P=0.006). The area under the concentration curve (AUC) and the incremental AUC (IAUC) was greater (P=0.020 and P=0.006). This difference may relate to the different chemical composition and possibly the presence of the glycolipids where the presence of DHA in krill oil limits the incorporation of EPA into plasma lipids. Also, the n-3 polyunsaturated fatty acids within glycolipids as found in the algae oil, but not in a krill oil, may be an effective system for delivering EPA to humans.

The incorporated by reference '037 patent describes the benefit of using an algae based oil, and more particularly, a marine based algae oil and discloses different manufacturing and production techniques. Microalgae can be cultured photoautotrophically outdoors to prepare concentrated microalgae products containing Eicosapentaenoic acid (EPA) and Docosahexaenoic acid (DHA), which are the long-chain polyunsaturated fatty acids (PUPAS) found in fish oil. Both are very important for human and animal health. The concentrated microalgae products as disclosed in the '037 patent may contain EPA and DHA and lipid products containing EPA and DHA purified from microalgae. The concentrated microalgae composition may be prepared by cultivating microalgae photoautotrophically outdoors in open ponds under filtered sunlight in a continuous or batch mode and at a dilution rate of less than 35% per day. The microalgae may be harvested in the exponential phase when the cell number is increasing at a rate of at least 20% of maximal rate. In one example, the microalgae is concentrated. In another example, at least 40% by weight of lipids in the microalgae are in the form of glycodiacylglycerides, phosphodiacylglycerides, or a combination thereof and at least 5% by weight of the fatty acids are DHA, EPA, or a combination thereof.

In one example, the microalgae are Tetraselmis sp. cultivated at above 20° C. or in another example at above 30° C. The EPA yield in the microalgae has been found to be at least 10 mg/liter culture. The microalgae can be Isochrvsis sp. or Pavlova sp. in another example, or are Thalassiosira sp. or Chaetecoros sp. The microalgae may be different diatoms and are cultivated photoautotrophically outdoors in open ponds for at least 14 days under filtered sunlight and at least 20% by weight of the fatty acids are EPA.

The use of this algae based oil overcomes the technical problems associated with the dwindling supplies of fish oil and/or Antarctic krill, which are now more difficult to harvest and obtain and use economically because these products are in high demand. A major difference between fish oils and algae based oils is their structure. Fish oils are storage lipids and are in the form of triacylglycerides. The algae based oils as lipids are a mixture of storage lipids and membrane lipids. The EPA and DHA present in algae based oils is mainly in the form of glycolipids and a small percentage is in the form of phospholipids. Glycolipids are primarily part of chloroplast membranes and phospholipids are part of cell membranes.

The '037 patent describes various methods for culturing microalgae photoautotrophically outdoors to produce EPA and DHA. One method used is filtering sunlight to reduce the light intensity on the photoautotrophic culture. Shade cloth or netting can be used for this purpose. It was determined that for most strains, the optimal solar intensity for growth, for maintaining a pure culture, and for omega-3 fatty acid accumulation was about 40,000 to 50,000 lux, approximately half of the 110,000 lux of full sunlight. Shade cloth or netting is suitable for filtering the sunlight to the desired intensity.

It is also possible to culture microalgae photoautotrophically outdoors and produce EPA and DHA by using small dilutions and a slow dilution rate of less than 40% per day, preferably less than 35% per day, more preferably from about 15% to about 30% per day. In other examples, the dilution rate is 15-40% per day or 15-35% per day, and in yet other examples, the dilution rate is 10-30%, 10-35%, or 10-40% per day. These smaller dilutions and lower dilution rates than are usually used help prevent contamination in outdoor photoautotrophic cultures. It also promotes thick culture growth that gives good DHA or EPA yield.

Another technique to successfully culture microalgae photoautotrophically outdoors and produce EPA and EPA/DHA is to harvest the microalgae in exponential phase rather than stationary phase. Harvesting in exponential phase reduces the risk of contamination in outdoor photoautotrophic cultures and has surprisingly been found to give a good yield of EPA and DHA. To drive fat accumulation in microbial cultures, the cultures are harvested in stationary phase because cells in the stationary phase tend to accumulate storage lipids. The '037 patent teaches that EPA and DHA accumulate in large amounts as membrane lipids in cultures harvested in the exponential phase. The membrane lipids containing EPA and DHA are predominantly phosphodiacylglycerides and glycodiacylglycerides, rather than the triaclyglycerides found in storage lipids. These cultures are harvested often when cell number is increasing at a rate at least 20% of the maximal rate, i.e., the maximal rate achieved at any stage during the outdoor photoautotrophic growth of the harvested culture. In specific examples, the cultures are harvested in exponential phase when cell number is increasing at a rate of at least 30%, at least 40%, or at least 50% of maximal rate. It is also possible to use recombinant DNA techniques.

The '037 patent includes several examples, which are referenced to the reader for description and teaching purposes.

### Example 1

The strain Thalassiosira sp. is a diatom and this strain used was isolated from Bay of Bengal, and it dominates during summer months. This example strain was isolated from seawater collected near Chemai, India, and the culture was maintained in open tubs. The particular strain was identified as Thalassiosira weissflogii, which is capable of growth at high temperatures (35-38° C.). The fatty acid profile was good even when the alga was grown at high temperature with 25-30% EPA (as a percentage of fatty acids).

Culturing:

The lab cultures were maintained in tubs in an artificial seawater medium, under fluorescent lights (3000-4000 lux) and the temperature was maintained at 25° C. Initial expansion of the culture was done under laboratory condition in tubs. The dilution rate was 15% to 30% of the total culture volume per day. Once the volume was 40-50 liters, it was transferred to an outdoor pond. The outdoor ponds were covered with netting to control the light (40,000 to 50,000 lux). The dilution continued until the culture reached 100,000 liters volume. The culture was held in 500 square meter ponds at this time with a culture depth of 20 cm. The culture was stirred with a paddle wheel and CO2 was mixed to keep the culture pH neutral. When the EPA levels in the pond reached a desirable level (10-15 mg/lit), the whole pond was harvested by filtration. The filtered biomass was washed with saltwater (15 parts per thousand concentration) and then spray dried. The mode of culturing was batch mode. The EPA productivity was 2-3 mg/lit/day. The ponds can also be run continuously for several weeks by harvesting part of the culture, recycling the filtrate into the ponds and replenishing required nutrients.

### Example 2

The strain Tetraselmis sp. is in the division Chlorophyta and the class Prosinophyceae or Micromanadophyceae. This strain was obtained from the Central Marine Fisheries Research Institute, India. It was isolated from the local marine habitats in India. The culture was maintained in flasks in artificial seawater medium, and expanded as described for Thalassiosira. With culture outdoors in open ponds as described for Thalassiosira, the strain gave a good lipid yield (200-300 mg/liter) and an EPA content of 6-7% of fatty acids.

### Example 3

The strain Chaetoceros sp. is another diatom strain obtained from the Central Marine Fisheries Research Institute, India, and isolated from local marine habitats in India. Chaetoceros sp. was maintained in flasks and cultivated in outdoor ponds photoautotrophically as described in Example 1. It gave similar EPA productivity and EPA content as Thalassiosira as described in Example 1.

### Example 4

The strain Isochrysis sp. is in the Prymnesiophyta, class Prymnesiophyceae, order Isochrysidales. It was obtained from the Central Marine Fisheries Research Institute, India, and isolated from local marine habitats in India. It was maintained and grown as described in Example 1. It was expanded from laboratory culture to a 50,000 liter outdoor pond culture in 14-15 days with a dilution rate of 15-30% per day. The lipid content at harvest was 100-150 mg lipids/liter. The rate of lipid production was 25-50 mg/liter/day. DHA was 10-12% of total fatty acids.

### Example 5

Harvesting and Drying:

The harvesting may be done by flocculation. The commonly used flocculants include Alum with polymer and FeCl3 with or without polymer and chitosan. The concentration of flocculent will depend on the cell number in the culture before harvest. The range may vary from 100 ppm to 500 ppm. Alternatively, harvesting is done by filtration using appropriate meshes. Removal of adhered chemicals (other than salt) is accomplished by washing the cells in low salinity water.

The harvested slurry is then taken for spray drying. The slurry is sometimes encapsulated to prevent oxidation. The concentration of encapsulating agent may vary from 0.1 to 1.0% on a dry weight basis. Modified starch is a suitable encapsulating agent. The spray dryer is usually an atomizer or nozzle type. The inlet temperature ranges from 160 to 190° C. and the outlet temperature ranges from 60 to 90° C. The spray dried powder is used immediately for extraction. If storage is required, the powder is packed in aluminum laminated pouches and sealed after displacing the air by nitrogen. The packed powder is stored at ambient temperature until further use.

### Example 6

Extraction of EPA/DHA is carried out using a wet slurry or dry powder and solvents, which include hexane, ethanol, methanol, acetone, ethyl acetate, isopropanol and cyclohexane and water, either alone or in combination of two solvents. The solvent to biomass ratio depends on the starting material. If it is a slurry, the ratio is 1:2 to 1:10. With a spray dried powder, on the other hand, the ratio is 1:4 to 1:30. The extraction is carried out in an extraction vessel under inert atmosphere, with temperature ranges from 25 to 60° C. and with time varying from one hour to 10 hours. Solvent addition is made one time or in parts based on the lipid level in the cells.

After extraction of crude lipid, the mixture is passed through a centrifuge or filtration system to remove the cell debris. The lipid in the filtrate is concentrated by removing the solvent by distillation, which is carried out under vacuum. The resulting product is a crude lipid extract, which contains approximately 10% omega-3 fatty acid (EPA/DHA). The extract can be used as it is or purified further to enrich the omega-3 fatty acids. Further purification may involve removal of unsaponifiables such as pigments, sterols and their esters.

It is also possible to use a pure diol of the S, S′astaxanthin. It is possible to use that pure diol in combination with the EPA rich algae based oil as described above and which is admixed with either astaxanthin derived from Haematococcus pluvialis or the free diol form in substantially pure S,S′ enantiomer form. It is possible to add synthetically derived mixed enantiomers of the diol as a product that is sold as a fish food in one non-limiting example. The diol of the S, S′astaxanthin is possible because in both cases of krill oil and possibly the algae based oil and Hp derived, there are principally diesters and monoesters respectively with very little diol, which is insoluble. Some research indicates that it may be many times more bioavailable than either the monoester or diester form. It is possible to asymmetrically synthesize the S,S′ pure diol. Despite the pure diol's poor solubility in some examples, there may be an active transport mechanism related to its bioavailability, or conversely, that only in the diol form is the monoester or diester forms transferred from the intestines to the blood. The phospholipid or glycolipid based product presenting EPA and/or DHA along with the added astaxanthin in its various forms and especially the S,S′ enantiomeric form in principally monoester form from Haematococcus pluvialis or pure dial form from asymmetric synthesis could be viable. Thus, it is possible to combine it with the algae derived glycol and phospholipid based EPA rich oil.

The composition may have other uses besides the primary use indicated above of improving blood lipid profiles and reducing low density lipoprotein (LDL) per-oxidation in humans.

Possible uses of the composition include use as a treatment for depression that may counter neurological disorders associated with depression. This could include treatment for a deficiency of neurotransmitters at post-synaptic receptor sites. The composition may be used to treat manic episodes in bipolar treatments and treat panic disorder and reduce the frequency and severity of panic attacks and the severity of agoraphobia. The composition may be used to treat Obsessive Compulsive Disorder (OCD) and malfunctioning neurotransmitters and serotonin receptors. The composition may also be used in the treatment of Alzheimer's Disease (AD) and reduce the presence of aluminosilicates at the core of senile plaque and diseased neurons. The composition may be used to treat aging disorders for cellular differentiation, proliferation and regeneration. It may also be used to treat age-related changes in mitochondrial function and age-related hearing loss. The composition may also possibly maintain metabolic activity and available energy by maintaining levels of phospholipids in normal cells and maintain membrane integrity and regulate enzyme activities and membrane transport processes through changes in membrane fluidity.

The composition may be beneficial for biological functions of essential fatty acids, including neural tissues such as the brain and retina and treat dementia-related diseases to increase mental function, memory, concentration and judgment and overcome the effects of Alzheimer's Disease. The composition may also be used to restore and preserve liver function and protect the liver against damage from alcoholism, pharmaceuticals, pollutant substances, viruses and other toxic influences that may damage cell membranes. It may possibly have antioxidant activity.

Additives may be used with the reaction composition and pharmaceutical or nutraceutical formulations may be made by methods known in the art. For example, the composition may be formulated in a conventional manner using one or more pharmaceutically or nutraceutically acceptable carriers. Thus, the composition may be formulated for oral administration. For oral administration, the pharmaceutical or nutraceutical compositions as compositions may take the form of, for example, tablets or capsules prepared by conventional techniques with pharmaceutically or nutraceutically acceptable excipients such as binding agents (e.g., pregelatinised maize starch, polyvinylpyrrolidone or hydroxypropyl methylcellulose); filters (e.g., lactose, microcrystalline cellulose or calcium phosphate); lubricants (e.g., magnesium stearate, talc or silica); disintegrants (e.g., potato starch or sodium starch glycollate); or wetting agents (e.g., sodium lauryl sulphate). The tablets may be coated by methods known in the art. Liquid preparations for oral administration may take the form of, for example, solutions, syrups or suspensions, or they may be presented as a dry product for use with water or another suitable vehicle before use. Such liquid preparations may be prepared by conventional techniques with pharmaceutically or nutraceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

When the composition is used as a nutraceutical, can be in the form of foods, beverages, energy bars, sports drinks, supplements or other forms as known in the art. This composition is also useful in cosmetic preparations, e.g., moisturizing creams, sun-block products and other topical cosmetic products as known in the art.

The composition may possibly be used in the treatment or prevention of a variety of disease states including: liver disease; chronic hepatitis; steatosis; liver fibrosis; alcoholism; malnutrition; chronic parenteral nutrition; phospholipid deficiency; lipid peroxidation; disarrhythmia of cell regeneration; destabilization of cell membranes; coronary artery disease caused by hypercholesterolemia; high blood pressure; menopausal or post-menopausal conditions; cancer, e.g., skin cancer; hypertension; aging; benign prostatic hyperplasia; kidney disease; edema; skin diseases; gastrointestinal diseases; peripheral vascular system diseases (e.g. leg ulcers); pregnancy toxemia; and neurodegenerative and psychiatric diseases (e.g. Parkinson's, Alzheimer's, autism, attention deficit disorder, learning disorders, mood disorders, bipolar depression, multiple sclerosis, muscular dystrophy).

The composition may also be useful for targeting tumors and may be used in conjunction with radioisotopes for diagnosing central nervous system tumors. The composition may also be used to reduce local fat deposits and reducing visible cellulite. The composition may also be used in aesthetics such as breast enlargement by acting on the lobular tissue of the breast and by increasing hydration of the breast.

The composition may be used to treat and/or prevent cardiovascular diseases, arthritis, skin cancer, diabetes, premenstrual syndrome and transdermal transport enhancement. It may be used to decrease cholesterol in vivo and inhibit platelet adhesion and plaque formation and reduce vascular endothelial inflammation in a patient and offer hypertension prophylaxis. The composition may prevent oxidation of low-density lipoprotein and have an inhibitory effect on the secretion of VLDL possibly due to increased intracellular degradation of APO B-100. It may offer a post-myocardial infarction prophylaxis possibly because of its ability to decrease CIII apolipoprotein B, to decrease C3 non-apoliproprotein B lipoproteins and to increase antithrombin 3 levels. It may be suitable for prophylactic usage against cardiovascular disease in humans where it relates to coronary disease, hyperlipidemia, hypertension, ischemic disease such as relating to angina, myocardial infarction, cerebral ischemia, and shock without clinical or laboratory evidence of ischemia or arrhythmia.

The composition may be suitable to offer symptomatic relief for arthritis, Still's Disease, polyarticular or pauciarticular juvenile rheumatoid arthritis, rheumatoid arthritis, osteoarthritis, and may provide clinical improvement in decreasing the number of tender joints and analgesics consumed daily by decreasing the production of interleukin and interleukin-1 in human patients. The composition may also be used as a skin cancer prophylactic. It may have some retinal and anti-carcinogenic effects. It may enhance transdermal transportation as a substrate for dermatological topical therapeutic applications and may be used in dermatological treatments via creams, ointments, gels, lotions and oils and may be used in various therapeutic applications such as relating to anesthesic, corticosteroids, anti-inflammatory, antibiotic and ketolytic functions. It may also be used to enhance transdermal transportation as a substrate for dermatological topical cosmetic applications where cosmetic application relates to skin hydration, anti-wrinkle, caratolytics, peeling and mask via creams, ointments, gels, lotions or oils. The composition may be used to reduce the pain and mood changes associated with premenstrual syndrome in women.

The composition may be used to treat or prevent a cardiometabolic disorder/metabolic syndrome. The cardiometabolic disorder could be atherosclerosis, arteriosclerosis, coronary heart (carotid artery) disease (CHD or CAD), acute coronary syndrome (or ACS), valvular heart disease, aortic and mitral valve disorders, arrhythmia/atrial fibrillation, cardiomyopathy and heart failure, angina pectoris, acute myocardial infarction (or AMI), hypertension, orthostatic hypotension, shock, embolism (pulmonary and venous), endocarditis, diseases of arteries, the aorta and its branches, disorders of the peripheral vascular system (peripheral arterial disease or PAD), Kawasaki disease, congenital heart disease (cardiovascular defects) and stroke (cerebrovascular disease), dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

The composition may also be used to treat, prevent or improve cognition and/or a cognitive disease, disorder or impairment (memory, concentration, learning (deficit)), or of treating or preventing neurodegenerative disorders. The cognitive disease, disorder or impairment could be Attention Deficit Disorder (ADD), Attention Deficit Hyperactivity Disorder (ADHD), autism/autism spectrum disorder (ASD), (dyslexia, age-associated memory impairment and learning disorders, amnesia, mild cognitive impairment, cognitively impaired non-demented, pre-Alzheimer's disease, Alzheimer's disease, epilepsy, Pick's disease, Huntington's disease, Parkinson disease, Lou Gehrig's disease, pre-dementia syndrome, Lewy body dementia dementia, dentatorubropallidoluysian atrophy, Freidreich's ataxia, multiple system atrophy, types 1, 2, 3, 6, 7 spinocerebellar ataxia, amyotrophic lateral sclerosis, familial spastic paraparesis, spinal muscular atrophy, spinal and bulbar muscular atrophy, age-related cognitive decline, cognitive deterioration, moderate mental impairment, mental deterioration as a result of ageing, conditions that influence the intensity of brain waves and/or brain glucose utilization, stress, anxiety, concentration and attention impairment, mood deterioration, general cognitive and mental well being, neurodevelopmental, neurodegenerative disorders, hormonal disorders, neurological imbalance or any combinations thereof. The cognitive disorder may be memory impairment.

The composition may be used to inhibit, prevent or treat inflammation or an inflammatory disease. The inflammation or inflammatory disease may be due to organ transplant rejection; reoxygenation injury resulting from organ transplantation (see Grupp et al., J. Mol. Cell. Cardiol. 31: 297-303 (1999)) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases (IBD) such as ileitis, ulcerative colitis (UC), Barrett's syndrome, and Crohn's disease (CD); inflammatory lung diseases such as asthma, acute respiratory distress syndrome (ARDS), and chronic obstructive pulmonary disease (COPD); inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, epilepsy, amyotrophic lateral sclerosis and viral or autoimmune encephalitis, preeclampsia; chronic liver failure, brain and spinal cord trauma, and cancer. The inflammatory disease may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer. Other disorders include depression, obesity, allergic diseases, acute cardiovascular events, muscle wasting diseases, and cancer cachexia. Also, inflammation that results from surgery and trauma may possibly be treated.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A dietary supplement composition to treat low density lipoprotein (LDL) oxidation in humans, wherein the dietary supplement composition comprises a therapeutic amount of algae based oil in combination with astaxanthin derived from Haematococcus pluvialis (Hp) in an oral dosage form, wherein the astaxanthin derived from Haematococcus pluvialis (Hp) is 0.1 to 2.7 percent by weight of the algae based oil.

2. The composition according to claim 1, wherein the astaxanthin derived from Haematococcus pluvialis (Hp) is 0.4 to 0.67% by weight of the algae based oil.

3. The composition according to claim 1, wherein the algae based oil comprises Eicosapentaenoic (EPA) and Docosahexaenoic (DHA) fatty acids in the form of glycolipids and phospholipids.

4. The composition according to claim 1, wherein the algae based oil comprises 5 to 10 percent phospholipids and 35 to 40 percent glycolipids.

5. The composition according to claim 1, wherein the algae based oil includes at least 15 percent EPA fatty acids.

6. composition according to claim 5, wherein the EPA fatty acids are conjugated with phospholipid and glycolipid polar lipids.

7. The composition according to claim 1, wherein the algae based oil is derived from the microalgae Nannochloropsis oculata comprising Eicosapentaenoic (EPA) fatty acids in the form of glycolipids and phospholipids.

8. The composition according to claim 1, wherein the algae based oil is derived from the microalgae selected from the group consisting of thalassiosira sp., tetraselmis sp., chaetoceros sp., and isochrysis sp., and comprising Eicosapentaenoic (EPA) and Docosahexaenoic (DHA) fatty acids in the form of glycolipids and phospholipids.

9. The composition according to claim 1, wherein the algae based oil is derived from the microalgae selected from the group consisting of grateloupia turuturu; porphyridium cruentum; monodus subterraneus; phaeodactylum tricornutum; isochrysis galbana; navicula sp.; pythium irregule; nannochloropsis sp.; and nitzschia sp. And comprising Eicosapentaenoic (EPA) and Docosahexaenioic (DHA) fatty acids in the form of glycolipids and phospholipids.

10. The composition according to claim 1, wherein the algae based oil is derived from the microalgae selected from the group consisting of Asterionella japonica, Bidulphia sinensis, Chaetoceros septentrionale, Lauderia borealis, Navicula biskanteri, Navicula laevis (heterotrof.), Navicula laevis, Navicula incerta, Stauroneis amphioxys, Navicula pellicuolsa, Bidulphia aurtia, Nitzschia alba, Nitzschia chosterium, Phaeodactylum tricornutum, Phaeodactylum tricornutum, Skeletonema costatum, Pseudopedinella sp., Cricosphaera elongate, Monodus subterraneus, Nannochloropsis, Rodela violacea 115.79, Porphyry. Cruentum 1380.Id, Pavlova salina, Cochlodinium heteroloblatum, Cryptecodinium cohnii, Gonyaulax catenella, Gyrodinium cohnii, Prorocentrum minimum, Chlorella minutissima, Isochrysis galbana ALII4, Phaeodactylum tricornutum WT, Porphyridium cruentum, and Monodus subterraneus and comprising Eicosapentaenoic (EPA) and Docosahexaenoic (DHA) fatty acids in the form of glycolipids and phospholipids.

11. The composition according to claim 1, wherein the algae based oil is derived from a fungi selected from the group consisting of Mortierella alpine, Mortierella alpine IS-4, and Pythium irregulars, or a bacteria as SCRC-2738 and comprising Eicosapentaenoic (EPA) and Docosahexaenoic (DHA) fatty acids in the form of glycolipids and phospholipids.

12. The composition according to claim 1, wherein the composition is formulated to deliver 1-4000 mg of algae based oil per daily dose.

13. The composition according to claim 1, wherein the composition includes algae based oil supplemented with 0.1-12 mg astaxanthin per daily dose.

14. The composition according to claim 1, wherein the astaxanthin is derived from Haematococcus pluvialis algae oleoresin or beadlet.
